(19) United States
(12) Patent Application Publication
(10) Pub. No.: US 2005/0170020 A1
(43) Pub. Date: Aug. 4, 2005

(54) Using organic and / or inorganic potassium and its salts to treat autoimmune and other health disorders and methods of administering the same

(76) Inventors: Munisekhar Medasani, Hyderabad (IN); Chandrasekhar Jonnalagadda, Houston, TX (US)

Correspondence Address: Ramesh Babu Vishwanath;# 48 SRT, 1st Floor, Prakash Nagar, Begumpet, Hyderabad, 500016, IN

(21) Appl. No.: 10/854,192

(22) Filed: May 27, 2004

Related U.S. Application Data
(60) Provisional application No. 60/474,181, filed on May 29, 2003.

(51) Int. Cl. A61K 35/78

(52) U.S. Cl. 424/722; 424/777; 424/757; 424/764; 424/735; 424/736; 424/766; 424/727

(57) ABSTRACT

A composition of potassium derived from organic source, preparation, method and amount of administration for treatment of autoimmune disorders and supplementation in the form of general preparation. A food source which is high in a natural or organic potassium content is first dehydrated to remove water to a substantial degree, i.e. freeze dried; the so dehydrated food source is then reduced to small particles and the carbohydrate content thereof is extracted there from by a solvent in which carbohydrates are more soluble but proteins and organic potassium compounds are not, such as aqueous ethanol; the residue that remains after carbohydrate extraction is dried of solvent and used in pharmaceuticals, food supplements, food products and cosmetics to supplement the body's intake of potassium without possible side effects.

### CROSS REFERENCE TO THE RELATED APPLICATIONS

The inventor herein has realized the effect of potassium deficiency on autoimmune disorders like psoriasis and started taking potassium in organic form to saturate the total body potassium levels. Once it started working for them, they started searching for any products and logics available to support their finding. They came across a US Patent filed by Oge, et al (U.S. Pat. No. 5,955,067) in which Oge, et al described the clearance of psoriasis by using potassium rich tube feeding formula with hospitalized patients which due to various medical surgical or neurological impairments lost their ability to receive oral feedings. Oge, et al also describe clinical trials on otherwise healthy individuals in controlled conditions to saturate total body potassium level. May be in view of problems associated with the hyperkalemia Oge, et al suggested attaining local saturation of potassium levels at the effected area of the skin by topical intradermic route.

### STATEMENT REGARDING FEDERALLY SPONSORED RESEARCH OR DEVELOPMENT

NA

### REFERENCES

1. Low Potassium Article by Prem C Shukla, MD in eMedicine, last updated May 28,
2. Potassium Metabolism, Nutrient-Drug Interactions etc from Merck Manual of Diagnosis and Therapy.
3. Homeostatic Adaptation to Reduced K diet without fall in plasma K. By J. P. Guzman, R. Angles, P. Leong, J. Youn and A. McDonough, University of South California, 2002, Li-cor Inc. http://bio. licor.com/Posters/552/552 conclusions.html
4. Hypokalemia Article by Eleanor Lederer in eMedicine, last updated May 28, 2001
5. Potassium Balance by Dr. T. Dixon (http://www.uhmc.sunysb.edu/internalmed/nephro/webpages/Part D. htm)
6. G G Krishna et al, Increased blood pressure during potassium depletion in normotensive men, The New England Journal of Medicine, Volume 320: 1177-1182 May 4, 1989 Number 18
7. The Bananas by James K Palmer, Food Science and Technology, The Biochemistry of Fruits and their products, Vol-2, 1971, Academy Press
8. F. J. Ebling, Skin Physiology as a basis for Cosmetic Practice, Hand Book of Cosmetic Science, 1963, Pergamon Press
9. Anthony du Vivier et al, Tachyphylaxis to the Action of Topically Applied Corticosteroids, Arch Dermatol/Vol 111, May 1975 pp. 581-583.
10. Mitsuhiro Denda et al, Low humidity stimulates epidermal DNA synthesis and amplifies the Hyperproliferative response to barrier disruption: Implication of Seasonal exacerbations of inflammatory dermatoses, The Society of Investigative Dermatology, Inc, 1998
11. M. W. Stanier et al, Energy balance and Temperature regulation, 1984, Cambridge University Press.
12. Mark Lyte, Induction of Gram-negative bacterial growth by neurochemical containing banana (Musa X paradisiacal) Extracts, 1997, ELSEVIER
13. Amy B. MacDermott et all, Cold Emerging from the Fog, Nature Neuroscience, vol. 5-3, March 2002.
14. Félix Viana et al, Specificity of cold thermotransduction is determined by differential ionic channel expression, Nature Neuroscience, vol. 5-3, march 2002
15. F. Maingret et al, TREK-1 is a heat activated background K⁺ Channel, The EMBO Journal, Vol. 19-11, 2000
16. M. Campero et al, Slowly conducting afferents activated by ionnocuous low temperature in human skin, Journal of Physiology, 2001, 535.3.
17. Christopher Miller, See potassium run, Nature, Vol. 414, 1 Nov. 2001
18. CJ Fowler et al, The conduction velocities of peripheral nerve fibres conveying sensation of warming and cooling, JNNP, 1988, Vol. 51.
19. Chirstopher Carruthers, Biochemistry of skin in health diseases, Charles C. Thomas Publisher, 1962
20. Alain Reinberg et al, BioChecmical changes in skin lesions and blood of psoriatic patients, Psoriasis, Charles C. Thomas Publisher, 1968
21. Edward C. Cooper et al, Ion cannel genes and human neurological disease: Recent progress, prospects and challenges, Proc. Natl. Acad. Sci, USA, vol 96, April 1999

### BACKGROUND OF THE INVENTION

The present invention relates to a use of organic forms of Potassium salts in a dietary supplement, and in pharmaceutical and cosmetic preparations that are effective for the treatment of autoimmune disorders like psoriasis, eczema, multiple sclerosis etc and other health disorders like potassium deficiency, hypertension, heart problem, kidney stones, cancer etc by elevating/correcting total body potassium levels and improving efficiency of Potassium pumps like Sodium-Potassium pumps etc of human body. This invention also relates to the use and method of administration of Banana peel extract, source of organic form of Potassium salts, alpha-adrenergic agonists (norepinephrine (NE)), vasoconstrictors and menthol in topical applications. This invention further relates to the use and method of administration of organic and inorganic forms of Potassium salts with alpha-adrenergic agonists (norepinephrine (NE)), vasoconstrictors and menthol in topical applications.

Potassium (K) is one of the minerals (also referred to as electrolytes) in the body. Potassium is the most abundant intracellular cation. Almost 98% of the potassium content of a healthy body is found inside the cells. Only about 2% of total body potassium is extracellular. Since most intracellular potassium is contained within muscle cells, total body potassium content is roughly proportional to lean body mass. An average 70-kg adult has about 3500 mEq of potassium.

Potassium is a major determinant of intracellular osmolality. The relationship between intra- and extracellular fluid potassium concentrations strongly influences cell membrane polarization, which in turn influences important cell processes, such as the conduction of nerve impulses and muscle (including myocardial) cell contraction. Thus, relatively small alterations in plasma potassium concentration can have major clinical manifestations.

Small changes in potassium concentration that is present outside the cells can have severe effects on the heart, nerves, and muscles. Potassium is important to mainta in several bodily functions.

Potassium is required to regulate pressure between the inside and outside of cells. The same will be done though Sodium-Potassium Pump. With inadequate potassium, cellular wastes are not efficiently transported into the extracellular spaces and carried away. Toxic material is left to accumulate in the cell can cause premature cell death.

Potassium is needed to convert blood sugar into glycogen for storage in the liver and muscles. With inadequate glycogen storage humans will not have strength and will quickly become exhausted physically and mentally.

Potassium is also required for pH balance of blood, body water balance for maintaining blood pressure. Potassium stimulates insulin production, digestive enzyme function and efficiency, nerve and muscle function. Potassium acts to relax muscle contraction In balance to calcium which induces contraction.

Many causes, such as utilization of medications like steroids, non-potassium sparing diuretics, some penicillins, exogenous bicarbonate ingestion, Amphotericin B, Gentamicin, physical and psychological stress, excess Sodium (Na) intake, can cause depletion of total body potassium levels.

With age, total body potassium level decreases. This decrease reflects the decrease in lean body muscle mass, which contains about 75% of intracellular potassium. Although aldosterone secretion decreases with age, the kidney's ability to regulate potassium excretion under normal dietary conditions is unaffected.

Most of the total body stores of potassium are within cells, so measurement of serum potassium is often inadequate for estimating total body potassium. Measuring urinary potassium excretion may help establish if urinary loss is abnormal. Urinary excretion of >20 mEq/L suggests an excessive loss in a patient with hypokalemia.

Regardless of the cause, hypokalemia produces similar signs and symptoms. Because potassium is overwhelmingly an intracellular cation and because a variety of factors can regulate the actual serum potassium concentration, an individual can incur very substantial potassium losses without exhibiting hypokalemia symptoms.

Normally, serum potassium levels will be maintained with minimum variation in the extracellular fluid (ECF), by drawing from the potassium stores of intracellular fluid (IFC). Because of this reason, unless there is severe deficit in total body potassium stores due to continuous deficit intake or loss of potassium from the body for various reasons, it will not reflect in the ECF and will be maintained in the normal range of 3.5-5 mEq/L.

As per approximate calculations, for every decrease in serum potassium of 1 mEq/L, the potassium deficit is approximately 200-400 mEq. However, many factors in addition to the total body potassium stores contribute to the serum potassium concentration. Therefore, this calculation could either be an overestimate or underestimate of the true potassium deficit.

When the dietary potassium intake falls, intracellular potassium again serves to buffer against wide swings in plasma potassium concentration. Renal potassium conservation develops relatively slowly in response to decreases in dietary potassium and is far less efficient than the kidneys' ability to conserve Na. Urinary potassium excretion of 10 mEq/24 h represents near maximal renal potassium conservation and, therefore, implies significant potassium depletion.

Inorganic form of potassium in tablet form containing more than 100 mg per dose cannot be taken without a doctor's prescription, as per US FDA guidelines, due to the side affects associated with sudden rise in serum potassium levels and GI track ulcerations. There are sustained and slow release tablets/capsules available, under doctor's prescription, to administer more than 100 mg of Potassium. But these are all associated with ulceration, in GI track. Other options available are Potassium Powders to be taken with liquids, and IV administration, which are mostly prescription medicines.

When doctors prescribe potassium supplements, it is usually in the range of 1,500 to 3,000 mg/day. When given in high-dose pill form, however, potassium salts can cause nausea, vomiting, diarrhea and ulcers.

It is already known and well documented that taking Potassium in natural forms such as raw vegetables and fruits will not cause side effects those associated with the oral form of inorganic potassium, even if taken at high doses. For example, a Banana contains about 400 mg potassium. In Africa banana is a staple food and personal consumption sometimes is about 35 cooked bananas per person per day (The Bananas by James K. Palmer, Food Science and Technology, The Biochemistry of Fruits and their products, Vol-2, 1971, Academy Press). There are baby food products and energy drinks, which contains banana and supplies 250-500 mg without causing any side effects. It shows that taking potassium in natural form at very high doses may not cause any hypokalemia.

Fruit drinks are available in the market, with exception to pure orange juice that are made from the concentrates and pulp and will contain 25-40% by weight pulp/volume. Banana powder is available in the market. But dry banana powder contains 88.28% of carbohydrates (USDA National Nutrient Database). Individuals who do not want to gain weight or who are on low carbohydrate diets do not prefer this high carbohydrate content. Bananas' in other than natural form are used for many home remedies, as detailed in the Medicinal Plants of the World, Vol-2, Page 320. The bananas are used in urban areas are mainly for the children who are suffering with diarrhea; to overcome dehydration. Bananas are also used in ready to serve foods for kids, in food preparations and as flavoring agent. Though banana drinks are available, but contains less of the natural fruit, more of added additives, excipients, preservatives and sugars, these may not be a good dietary source of potassium.

Raw Banana is used in Ayurveda Medication (Indian Traditional Medicine) for ages as an effective astringent and is recommended widely as a diet for treating diarrhea.

In Homeopathy they use Kali-arsenicum, Kali-bromatum, Kali-Sulphuricum which are potassium salts for treating psoriasis, but these are used along with elements such as sulphur, mercury. For some people homeopathy is effective in treating psoriasis, but for many it is difficult to follow the strict diet and alcohol restrictions they suggest.

Potassium works synergistically with sodium in the body. However our typical intake of potassium vs. sodium is considered wrong. A fresh fruit and vegetable diet has a hundred times more potassium than sodium. Researchers recommend an intake of at least 5 times more potassium than sodium. As per the Article in The American Journal of Clinical Nutrition (Am J Clin Nutr 2000; 71: 1020-6), Late Paleolithic diets used to contain 6,970 mg of Potassium and 604 mg of Sodium, which is a 12:1 ratio. Unfortunately, most modern diets have so much salt that they reverse the ratio with Potassium: Sodium being about 0.7:1. Added salt is 95% of our dietary sodium. An Average American diet consists of 3,400 mg of Sodium against minimum requirement of 500 mg and 2,400 mg of Potassium against a requirement of 3,500 mg. Most of our convenience foods have added salt (sodium chloride), monosodium glutamate (MSG) for taste purpose. The body expects abundant potassium and less sodium.

Eating vegetables and fruits in raw form is the best way of getting the potassium required for the body. Excess potassium cannot be stored in the body, excess potassium will be excreted in 1-2 hrs to maintain the proper serum potassium level. It is know that stress depletes potassium from the body. Stress can take many forms: taking an examination, recovering from a broken bone, or maintaining proper levels of energy substrates in the face of even mild starvation. For human males, there is even considerable stress associated with shopping. To get enough potassium as required the food pyramid guidelines suggests eating 5-9 servings per day of fruits and vegetables.

Though such a high fruits and vegetables diet is healthy, many people do not follow this, which may be because this is not convenient, consumes time and for some people eating raw fruits/vegetables causes stomach gas and feels uncomfortable in public places. Also many fruits and vegetables which are good sources of potassium like avocado, banana, potato, dates etc are high in carbohydrates which could be one of the reasons people do not take these on a regular basis.

### SUMMARY OF THE INVENTION

It is evident that body potassium levels will deplete due to many reasons, which also includes stress. With urbanization, unhealthy lifestyles and world economic conditions, stress is increasing for human beings, which causes depletion of body potassium levels on a continuous basis, causing health problems. Stress can potentially contribute to many diseases like high blood pressure, diabetes, heart problems, cancer, GI track acidity and ulceration, autoimmune disorders like psoriasis, eczema etc. To make the situation worse, convenience foods have more sodium and minimum/ml potassium.

Due to the adverse reaction associated with the oral intake of inorganic potassium and as per the FDA guidelines, 99 mg per tablet is not enough to replenish body potassium loss on a ‘regular basis’ unless one adopts to a regular intake of fresh fruits and vegetables.

This invention provides convenience and a less time consuming way of administration of more than 100 mg of Potassium (organic and inorganic) without possible side effects associated with the present oral dosages, with the administration being in the form of a food supplement, pharmaceutical or cosmetic.

Accordingly, a food source which is high in a natural or organic potassium content is first dehydrated in a known manner to remove water to a substantial degree, i.e. freeze dried; the so dehydrated food source is then reduced to small particles' and the carbohydrate content thereof is extracted there from by a solvent in which carbohydrates are more soluble but proteins and organic potassium compounds are not, such as aqueous ethanol; the residue that remains after carbohydrate extraction is dried of solvent and used in pharmaceuticals, food supplements, food products and cosmetics to supplement the body's intake of potassium without possible side effects. The potassium rich extract may be administered in the form of a powder, granules, tablets, caplets, capsule, Effervescent tablet, Effervescent powder, chewable tablet, chewing gum, drink mix, suspension, enteric coated tablets, enteric coated granules, sustained release tablets. A preferred food source for this operation is bananas or plantains, and/or their roots, pulp, peel, stalk, leaves, stem, suckers, flowers, where these are from the botanical family Musaceae of the order Zingiberales and its genera: Musa. Alternatively, the extraction potassium rich extract may be formulated into a pharmaceutical topical applications for treating autoimmune and other skin diseases like, but not limited to, psoriasis, atopic dermatitis, eczema. Alternatively, to administer potassium to the body any pharmaceutically acceptable potassium salt of organic or inorganic form may be fabricated into a transdermal patch for administering 100 to 7,000 mg per day and even more preferably 100 to 3,000 mg per day of potassium to the user.

The inventor herein has realized the effect of potassium deficiency on autoimmune disorders like psoriasis and started taking potassium in organic form to saturate the total body potassium levels. Once it started working for them, they started searching for any products and logics available to support their finding. They came across a US Patent filed by Oge, et al (U.S. Pat. No. 5,955,067) in which Oge, et al described the clearance of psoriasis by using potassium rich tube feeding formula with hospitalized patients which due to various medical surgical or neurological impairments lost their ability to receive oral feedings. Oge, et al also describe clinical trials on otherwise healthy individuals in controlled conditions to saturate total body potassium level. May be in view of problems associated with the hyperkalemia Oge, et al suggested attaining local saturation of potassium levels at the effected area of the skin by topical intradermic route.

The present invention differs from that of Oge, et al in its form of administration and its focus, i.e. attaining saturation of total body potassium levels on a regular basis. The present invention also works as a preventive measure for many autoimmune and other diseases.

### DETAILED DESCRIPTION OF THE PREFERRED EMBODIMENTS OF THE INVENTION

Inventor himself is a psoriasis patient for the last 9 years. He tried all topical applications like salicylic acid, coal tar, steroid based etc and experienced all sorts of side effects associated with these presently available treatments and also developed psoriatic arthritis for which doctors started administering intra muscular steroid injections to relieve the pain and swelling.

The inventor tried to understand regarding psoriasis, why psoriasis gets cured automatically for some people and why there is no identified cause for psoriasis when it is getting cured for some people, why there is no one universally accepted treatment for this problem. While trying to understand this problem, which they developed after moving to Dubai, UAE (which is desert climate) from tropical climatic places of South India, they noticed a coincidence among 5 different people who moved to Dubai with similar backgrounds, from same age group, with similar diet and living habits. Out of 5 people, 4 were affected with psoriasis, 3 with gouts, 2 with kidney stones, 2 with bone degeneration and 1 with hypertension. As all of them are inventor's family friends their eating and living habits were fully known, which might have gone unnoticed otherwise.

They noticed that for convenience, to reduce additional carbohydrates (as their basic diet was mainly of rice), to reduce fat intake, due to non-availability of a few food items, which they used to eat in India; they avoided the vegetables, fruits, and milk products, which the used to consume earlier. This had reduced their regular intake of potassium drastically when compared with their earlier intake. Due to usage of a few Calcium fortified products their calcium intake was being maintained, but potassium intake had come down substantially to create a depletion of total body potassium levels.

The inventor then started investigating about potassium mechanism in the human body, which includes, maintaining homeostasis, blood pH maintenance, carbohydrate and protein metabolisms, blood glucose regulation, glycogen synthesis, conduction of nerve impulses, muscle (including myocardial) cell contraction etc. They also noticed that there is no proper and commonly available test procedure to find out intracellular potassium levels. Urine excretion tests can also go wrong, as intake can vary between 50-125 mEq/day Serum potassium level can give wrong impression as the human body is equipped to maintain serum potassium levels within minor variation even if intracellular potassium is depleted.

If an experimental animal is maintained on a low or moderate potassium intake, a sudden increase in dietary potassium may result in severe hyperkalemia and the animal may die However, if the low potassium diet is gradually supplemented with additional potassium the same large potassium loads, which previously had produced dangerously high plasma potassium levels become harmless. The animal becomes adapted to high potassium loads through the process of ingesting gradually increasing amounts of potassium in its diet. The physiologic components of the adaptation include the ability to excrete a potassium load more quickly (renal potassium secretion rates are markedly enhanced) and the temporary storage of potassium in the intracellular fluid is more effective. Thus, following a large load of potassium, plasma potassium levels do not rise to the same degree in the potassium-adapted animal as they do in the non-adapted animal.

The mechanism(s) responsible for potassium adaptation are not well understood. There is evidence that diets high in potassium result in increased aldosterone secretion rates, increased insulin release, the induction of larger amounts of Na K ATPase in the cells of the renal tubule and the large intestine. It can be shown that the potassium secretion capacity of the distal nephron (specifically the distal half of the distal convoluted tubule and the cortical collecting tubule) is markedly enhanced in animals on high potassium intake and this enhancement can be shown to characterize the function of the isolated nephron segment in vitro as well as in vivo.

Though the mechanism which is affecting autoimmune disorders like psoriasis, eczema etc due to low total body potassium is not understood clearly, it was noticed that by increasing the daily intake of potassium and attaining the saturation of total body potassium levels the conditions of disease improved and also other conditions like gouts, bone degeneration, hypertension, insomnia etc the inventor was facing along with psoriasis were corrected.

Inventor has used organic form of potassium to attain the saturation of total body potassium, by gradually increasing the daily additional intake of potassium till it reaches about 2,500 mg/day. They have noticed that it took few months before they started seeing results like no gout attacks, no insomnia, started feeling strength in the muscles, no depressed feeling etc. Psoriatic flare-ups become less frequent. As they have not used any other regular psoriasis medications along with this, it took extra time before the body gets used to the changes and reactions become milder. Inventor who was suffered from both psoriasis and psoriatic arthritis it took about 10 months before his psoriatic arthritis affected joints become almost normal and he was able to use the same like his other normal joints. Also his shoulder and neck in which he used to have severe pain became normal and it never pained again.

Another person in the study group, whose backbone has developed a crack due to bone degeneration and who started facing severe problem with his daily life and work, has recovered in about 4 months and now he is able to perform his job in normal condition. After that he never faced any gout attacks, which were frequent earlier.

Inventor also noticed that, if they discontinued the additional potassium intake for more than few days, they started getting flare-ups of psoriasis and also joint pains and gout attacks.

With what he has done to increase the body level of potassium, good results he has noticed by keeping rest of the eating and living habits normal, he has come to following conclusions:
1. When potassium intake is increased gradually, body and kidneys are able to coup with the additional load of potassium and are able to maintain serum potassium balance efficiently without side effects.
2. Due to the problems associated with the oral doses of potassium, there should be some other mode of supplementing potassium on daily basis without problems associated with oral doses and without sudden increase of serum levels.
3. While attaining the saturation of potassium levels in the body, use of medicines like steroids, neurotransmitters, channel openers in mild dosages will speedup the recovery and reduction in allergic conditions.
4. Natural and organic form of potassium is the best way to supplement potassium in human body and potassium supplement products should be based on the natural form but without the high carbohydrates attached and without stomach gas discomforts.
5. The potassium supplement should be convenient and less time consuming to administer than the consumption of 5-9 servings per day of fruits and vegetables.

Accordingly, a food source which is high in a natural or organic potassium content is first dehydrated in a known manner to remove water to a substantial degree, i.e. freeze dried, the so dehydrated food source is then reduced to small particles and the carbohydrate content thereof is extracted there from by a solvent in which carbohydrates are more soluble but proteins and organic potassium compounds are not, such as aqueous ethanol, the residue that remains after carbohydrate extraction is dried of solvent yielding a potassium rich extract which is used in pharmaceuticals, food supplements, food products and cosmetics to supplement the body's intake of potassium without possible side effects. The potassium rich extract may be administered in the form of a powder, granules, tablets, caplets, capsule, Effervescent tablet, Effervescent powder, chewable tablet, chewing gum, drink mix, suspension, enteric coated tablets, enteric coated granules, sustained release tablets. A preferred food source for this operation is bananas or plantains, and/or their roots, pulp, peel, stalk, leaves, stem, suckers, flowers, where these are from the botanical family Musaceae of the order Zingiberales and its genera: Musa. Alternatively, the extraction potassium rich extract may be formulated into a pharmaceutical topical applications for treating autoimmune and other skin diseases like, but not limited to, psoriasis, atopic dermatitis, eczema. Alternatively, to administer potassium to the body any pharmaceutically acceptable potassium salt of organic or inorganic form may be fabricated into a transdermal patch for administering 100 to 7,0000 mg per day and even more preferably 100 to 3,000 mg per day of potassium to the user.

The potassium rich food source that may be used include, but is not limited to, bananas, avocados, orange, prunes, apricots, mangos, raisins, dates etc; from vegetables like potato, sweet potato, tomato, spinach etc; from seeds lima beans, fried beans, soya beans, sunflower seeds, almonds etc. With reference to bananas as an example, a 100 g banana comprises about 75.1 g water, 1.2 g protein, 0.3 g fat, 23.2 g carbohydrates and 400 mg potassium. After dehydration by freeze drying and removal of carbohydrates by extraction with aqueous ethanol, the dried residue which remains is a mass of about 1.7 g that mainly comprises the natural potassium salt and protein content of the original banana. The resulting potassium rich extract contains moisture from 3% to 15% and may be pulverized to particle size less than 250 microns. Consumption of about 10-50 g of such a potassium rich extract over the course of a day in the form of powder, granules, tablet, caplet, capsule, Effervescent tablet, Effervescent powder, chewable tablet, chewing gum, drink mix, suspension, enteric coated tablets, enteric coated granules, sustained release tablets, etc. supplies 3,000 mg per day of potassium to the user. As desired such potassium rich extract may be supplemented with vitamins, minerals, flavoring agents, fibers, coloring agents, and/or taste improvers. Such potassium rich extract may be formulated into a lotion, cream, ointment, emulsion, solution, patch, cleanser, conditioner, gel, soap, sprays, foam, cosmetic and tape which may then be applied as a topical application for treating autoimmune and other skin diseases like, but not limited to psoriasis, atopic dermatitis, eczema.

Such potassium rich extract may be formulated into a pharmaceutically acceptable transdermal patch for administering 100 to 7,000 mg per day and even more preferably 100 to 3,000 mg per day, by any pharmaceutically acceptable patch making technologies. When administration of the potassium is by a transdermal patch forms of potassium other than that obtained from such potassium rich extract may be used, such as inorganic potassium salts, homeopathic salts (like Kali-Sulphuricum etc), ayurvedic salts of potassium or a combination thereof.

Though the health problems have comedown substantially for all the members, still they have noted mild psoriasis flare-ups now and then. Their observation shown that they are getting flare-ups only when they are in dry conditions, like continuous exposure to the high air-conditioning which they faced during the long flight journeys, dry desert winter weather etc.

Having their own experiences in the mind, they have studied the medical and other literature available and their findings have summarized below:

Skin Functions in Protecting Body Fluids and Temperature:

The skin is structured to prevent loss of essential body fluids. The Adult Human body contains water at about 60% of body weight. Adult human skin contains about 60-65% of water. In the absense of stratum corneum we would all lose significant amounts of water to the environment, and rapidly become dehydrated. The skin is a vital part of the body's temperature regulation system, protecting us against hypothermia and hyperthermia.

There is a continuous loss of water from the skin even at low temperatures. Probably about ⅘^{th} of this outward transport of water takes place through the sweat glands, but the droplets are so small and evaporation so rapid that they cannot be seen with the naked eye. The reminding ⅕^{th} is lost transepidermally. Lipid soluble substances such as vitamins A and D, Steriod Hormones, Salicylic Acid etc penetrate the skin with ease. It is probable that the major pathway of this absorption is through the hair follicles and the sebaceous glands (F. J. Ebling).

Scalp consists of 5 layers of which the first three are the skin, connective tissue and epicranial aponeurosis. These three layers are bound together as single unit. Connective tissue (superficial fascia) provides a passage way for nerves and blood vessels. Blood vessels are attached to this fibrous connective tissue. If the vessels are cut, this attachment prevents vasospasm, which could lead to profuse bleeding after injury. Wounds in the scalp bleed profusely because the fibrous fascia prevents vasoconstriction.

The head and upper truck have more sebaceous glands than other parts of the body. Which coincides with Dr. Steven R. Feldman comment that the scalp psoriasis occurs in at least 50 percent of all people with psoriasis and dermatologists think scalp psoriasis is a special kind that won't go away easily, in The world conference on Psoriasis.

A previously unrecognized pharmacological event, acute tolerance to the vasoconstrictive action of topically applied glucocorticosteroids, has been discovered in human skin. Thus, potent topical glucocorticosteroids will cause vasoconstriction when first applied to human skin but with subsequent applications the production of vasoconstriction rapidly diminishes. However, after a rest period of a few days, the same Initial vasoconstrictive effect may be produced again, but this will also disappear if the steroid is again continued topically (Anthony du Vivier et al.).

Studies by Mitsuhiro Denda et al, indicate that exposure to changes in the environmental humidity alone induces increased keratinocyte proliferations and makers of inflammation and that these changes are attributable to changes in stratum corneium moisture content. Finally these studies provide evidence that changes in environmental humidity contribute to the seasonal exacerbations/amelioration of cutaneous disorders, such as atopic dermatitis and psoriasis, diseases which are characterized by a defective barrier, epidermal hyperplasia and inflammation. (Mitsuhiro Denda et al).

The above denotes why winter and cold conditions aggravate the psoriasis and how topically applied glucocorticoids are effective on the same. By vasoconstriction of blood vessels, glucocorticoids minimize the water loss from the skin.

Hairs grow out of tubular invaginations of the epidermis known as follicles, and a hair follicle and its associated sebaceous glands are referred to as a pilosebaceous unit. Hair follicles extend into the dermis at an angle. A small bundle of smooth muscle fibers, the arrector pili muscle, extends from just beneath the epidermis and is attached to the side of the follicle at an angle. Arrector pili muscles are supplied by adrenergic nerves, and are responsible for the erection of hair during cold or emotional stress (‘goose flesh’). The sebaceous gland is attached to the follicle just above the point of attachment of the arrector pili.(www.telemedicine.org).

In all species of mammals the hypothalamus is the principal region in the cetral nervous system where the afferent pathways from temperature sensors act upon efferent pathways to thermoregulatory effectors by which autonomic and somatic nevers and endocrine glands make appropriate responses. The anterior region of the hypothalamus is principally involved in the control of responses to the warm environment (sweating, increased skin blood flow) and that the posterior region is principally involved in the control of responses to cold (shivering, vasoconstriction). (M. W. Stanier et al).

Other finding by Suskind. R. R. (1954) and Christine Kronauer (2001) identified that one of the major functions of psoriatic lesion is preventing water loss and sweat retention in the body.

From above it is clear that by reducing the body water loss i.e. by vasoconstriction we can control the psoriasis. To check this theory inventor has applied Phenylephrine which is used for nasal decongestant, on psoriasis affected scalp area. Phenylephrine is an Adrenergic Agent that mimics the sympathetic neurotransmitter norepinephrine (NE). Psoriatic skin has responded well to that and lesions reduced. This worked by way of reducing water loss of psoriatic skin by vasoconstriction.

Inventor has noticed that there is high concentration of norepinephrine (NE) in the peel of banana (Mark Lyte). The extract of banana peel will be beneficial for treating skin diseases like psoriasis, atopic dermatitits, eczema etc by activating the cold nerves. Inventor found that using Banana peel extract or alpha-adrenergic agonist and/or Methol with Potassium Salts will activate the cold sensitivity and prevents the body water loss, which is one of the main triggers for psoriasis.

Inventor has come out with solutions, which can increase the body potassium levels to reach saturation levels without the side effects associated with the present available food supplements and medications and also few topical applications which can prevent the water loss from the human body.

### EXAMPLE: 1

Preparation of Potassium Rich Extract:

3.5 ltrs of filtered demineralised water was taken into a 5 ltrs reaction flask and 1 kg of dried banana powder was added into it. Stirred for one hour and soaked it overnight. After the soaking the mass was stirred for 6 hrs at 60-65 degrees C. and filtered. The filtrate was collected into a conical flask and labeled as Filtrate-A.

The solid part was taken back into the reaction flask and added 1.5 ltrs of demineralised water. Heated the mass under stirring to 60-65 degrees C. and stirring was continued to 3 hrs with the same temperature. Mass was filtered and the filtrate was collected in a conical flask and labeled as Filtrate-B.

The Filtrate A and B were taken into a distillation unit and distilled 75% of its volume under reduced pressure and temperature not exceeding 90 degrees C. 600 ml of alcohol was added and cooled to the room temperature to precipitate. The precipitate was filtered and washed the cake with aqueous alcohol. The filtrate and washing were collected together and evaporated to dryness. The dry residue was washed with alcohol till the material stickiness is lost and further dried in a dryer to yield 15 grams of dried potassium rich extract.

The dried potassium rich extract was analyzed for the potassium, total sugars and moisture were determined by Atomic Emission Spectrometry, HPLC using refractory index detector, desiccation at 105 degrees C. respectively. Results were as under:
Potassium content—11.25%
Total Sugars—23.70%
Moisture—1.62%

### EXAMPLE: 2

Preparation of Banana Peel Extract:

Ripped banana peel were taken without hard end parts and cut into small pieces of 200 grams. The small pieces were loaded to a juice extractor and extracted juice from the peel. The collected thick juice was strained through a filter cloth to get almost clear light brownish liquid of 80 ml. Added 0.1% methylparaben as preservative. The total solution was allowed to settle overnight. Decanted the supernatant liquid of 75 ml into a clean sterilized bottle, which was stored at 10 degrees C. for further formulation.

### EXAMPLE: 3

Preparation of Banana Peel Extract Cream:

Oil Phase

| S.no | Name of the material | Units | Qty |
|---|---|---|---|
| 01 | Stearic Acid | Gm | 12.00 |
| 02 | Glyceryl Monostearate | Gm | 8.00 |
| 03 | Cetyl alcohol | Gm | 2.00 |
| 04 | Iso Propyl Myristate | Ml | 12.0 |
| 05 | Propyl Propyl paraben | Gm | 0.10 |

In a 200 ml flask exact quantities of above ingredients were taken and heated up to 75-80° C. with slow stirring when the temperature reaches to 75-80° C., the solution becomes clear. Mean while the below mentioned water phase was prepared.

Water Phase

| S.no | Name of the material | Units | Qty |
|---|---|---|---|
| 01 | Glycerin | Ml | 10.50 |
| 02 | Tri ethanol amine | Ml | 4.00 |
| 03 | HPMC | Gm | 1.50 |
| 04 | Water | Ml | 40.0 |
| 05 | Ammonium chloride | Gm | 0.10 |
| 06 | Potassium chloride | Gm | 0.10 |
| 07 | Methyl parabin | Gm | 0.10 |

In a 200 ml flask exact quantities of above ingredients were taken and heated up to 75-80°c with sloe stirring when the temperature reaches to 75-80° C., the solution becomes clear.

The above two phases were poured into a mortar and mixed thoroughly with pestle to get uniform mixture and checked the pH and adjusted to 5-6 by adding buffering agent. When the temperature reaches to 40° C. a few drops of Vanilla extract, 20 ml of Banana peel extract and fragrance was added. The mixture was slowly cooled to room temperature. The cream was packed in to clean plastic tins.

### EXAMPLE 4

Preparation of Potassium Compound Transdermal 6 hr Patch:

Transdermal patches 5 cm in diameter is prepared for the delivery of potassium chloride. The patches are composed of a tri-laminate of an adhesive matrix sandwiched between an occlusive backing layer and a release liner. The adhesive matrix is prepared from the pressure sensitive silicone adhesive together with potassium chloride. The occlusive backing film is a polyester film (about 3.00 mil in thickness). The release liner is a polyester film (about 3.0 mil in thickness). The final transdermal patch is about 16 mil thick, 5 cm in diameter and has a surface area of about 10 cm. In use, the transdermal patch is applied to a patient by removing the release liner and contacting the adhesive unit with the skin to supply 300 mg of potassium in 6 hrs.

While certain novel features of this invention have been shown and described and mentioned in the claims, it is not intended to be limited to the details above, since it will be understood that various omissions, modifications, substitutions and changes in the forms and details of the device illustrated and in its operation can be made by those skilled in the art without departing in any way from the spirit of the present invention.

1. A composition of potassium derived from organic source, preparation, method and amount of administration for treatment of autoimmune disorders and supplementation in the form of general preparation.

2. As claimed in claim 1, wherein composition of matter comprises an extract, which is rich in natural organic potassium resulting from the process of a raw or dehydrated organic source, which includes a solvent extraction, filtration, evaporation and drying.

3. As claimed in claim 1, wherein the methods of administration of the potassium are oral, topical and transdermal patches.

4. As claimed in claim 1, wherein the organic sources are a potassium rich fruit, vegetable, seed. T

5. As claimed in claim 4, wherein the potassium rich fruit is selected from the group consisting of bananas, avocados, orange, prunes, apricots, mangos, raisins, dates, and kiwis.

6. As claimed in claim 4, wherein the vegetable is selected from the group consisting of potato, sweet potato, tomato, spinach.

7. As claimed in claim 4, wherein the seeds are selected from the group consisting of seeds lima beans, fried beans, soya beans, sunflower seeds, almonds.

8. As claimed in claim 5, wherein the bananas means its plantain, roots, pulp, peel, stalk, leaves, stem, suckers, flowers from the botanical family Musaceae of the order Zingiberales and its genera:Musa.

9. As claimed in claim 8, wherein the banana peel extract comprises mainly norepinephrine, potassium and plant sterols is for topical administration.

10. As claimed in claim 9, wherein the norepinephrine is used in the treatment of skin disorders like psoriasis, eczema, atopic dermatitis.

11. As claimed in claim 10, wherein the norepinephrine means it's organic and inorganic forms and their salts having property of alpha-adrenergic receptor, vasoconstrictor and avoiding water loss from the skin.

12. As claimed in claim 3, wherein the transdermal patch administration of potassium is along with other vitamins, minerals and a suitable carrier.

13. As claimed in claim 2, wherein dehydration is accomplished by freeze-drying or heat drying and during drying of said organic source, temperatures of the process do not exceed 90 degrees centigrade.

14. As claimed in claim 2, wherein solvent extraction is effectuated with one or more solvents selected from the group consisting of ammonia, benzene, ethanol, hexane, chloroform, methanol, acetone, butanol, methyl chloride, petroleum ether and water.

15. As claimed in claim 14, wherein solvent extraction is effectuated more preferably with aqueous ethanol.

16. As claimed in claim 2, wherein potassium rich extract is pulverized to a particle size of less than 250 Microns.

17. As claimed in claim 1, wherein the form of general preparation are food products, food supplements, pharmaceuticals and cosmetics.

18. As claimed in claim 1, where the amount of administration is between 100 to 7,000 mg per day of said potassium to the human body.

19. As claimed in claim 1, wherein composition is further fortified with vitamins, minerals, fibers, flavoring agents, coloring agents and taste enhancers.

20. As claimed in claim 1, wherein the autoimmune disorders are, but not limited to, psoriasis, eczema, atopic dermatitis, multiple sclerosis.

21. As claimed in claim 3, wherein the oral administration is accomplished in the form of powder, granules, tablet, caplet, capsule, effervescent tablet, effervescent powder, chewable tablet, chewing gum, drink mix, suspension, enteric coated tablets, enteric coated granules, sustained release tablets, food supplements, or food products.

22. As claimed in claim 3, wherein the topical administration is having potassium, alpha-adrenergic receptor agonist, vasoconstrictor, menthol and a suitable carrier as a composition.

23. As claimed in claim 3, 9 and 22, wherein topical administration is accomplished in the form of a lotion, cream, ointment, emulsion, solution, patch, cleanser, shampoo, conditioner, gel, soap, sprays, foam or tape applied to the affected area of the skin.

24. As claimed in claim 12 and 22, wherein the potassium is from organic and inorganic source.

25. As claimed in claim 24, wherein a inorganic potassium is selected from the group consisting potassium carbonate, potassium chloride, potassium citrate, potassium gluconate, potassium acetate, potassium bicarbonate, potassium aluminate, potasium iodate, potassium iodide, potassium manganate, potassium permanganate, potassium phosphate monobasic, potassium phosphate dibasic, potassium phosphate tribasic, potassium phosphite, potassium arsenite solution, potassium bisulfate, potassium bitartararte, potassium bromide, potassium glycerophosphate, homeopathic salts of potassium, ayurvedic salts of potassium, natural potash, chelated potassium or a combination thereof,

26. As claimed in claim 22, wherein the alpha-adrenergic receptor agonists is selected from the group consisting of norepinephrine, epinephrine.

27. As claimed in claim 22, wherein the vasoconstrictor is selected from the group consisting of oxymetazoline, xylometazoline, pseudoephedrine, ephedrine, naphazoline, tetrahydrozoline, isoproterenol, phenylephrine, methoxamine, clonidine and their salts.

28. As claimed in claim 9, wherein plant sterols comprising from the group of beta-sitosterol, campesterol, stigmasterol, daucosterol, sitoindosterol.

29. As claimed in claim 2, wherein the organic source leftover after the extraction, is further extracted to obtain a composition contains potassium, carbohydrates, dietary fiber, which is used in food and dietary supplement formulations.
